(19) United States
(12) Patent Application Publication
(10) Pub. No.: US 2002/0151930 A1
(43) Pub. Date: Oct. 17, 2002

(54) Acupressure and reflexology clamp

(76) Inventor: Jeremy Mills, Marietta, GA (US)

Correspondence Address: MYERS & KAPLAN, INTELLECTUAL, PROPERTY LAW, L.L.C., 1827 POWERS FERRY ROAD, BUILDING 3, SUITE 200,, ATLANTA, GA, 30339, US

(21) Appl. No.: 10/119,473

(22) Filed: Apr. 10, 2002

Related U.S. Application Data
(60) Provisional application No. 60/283,991, filed on Apr. 16, 2001.

(51) Int. Cl. A44B 21/00; A61B 17/00

(52) U.S. Cl. 606/204; 24/489

(57) ABSTRACT

An acupressure and reflexology clamping apparatus wherein a variety of interchangeable pressure delivery tips enables selection thereof for a variety of applications and body points and a centrally positioned ratcheting hub enables fine incremental adjustment of pressure settings, reproducible selection of such settings and consistent delivery thereof, thereby delivering incrementally variable quantities of pressure to specific body locations and maximizing the effective relief experienced at each such location.

### CROSS-REFERENCE TO RELATED APPLICATIONS

To the full extent permitted by law, the present application claims priority to and the benefit as a non-provisional application to provisional patent application entitled “Acupressure and Reflexology Clamp” filed on Apr. 16, 2001, having assigned Ser. No. 60/283,991, wherein said application is incorporated herein by reference.

### TECHNICAL FIELD

The present invention relates generally to acupressure devices and, more specifically, to an acupressure and reflexology clamping apparatus capable of delivering incrementally variable quantities of pressure to specific body locations and maximizing the effective relief experienced at each such location by utilizing a variety of easily interchangeable pressure delivery tips. The present invention is particularly useful in, although not strictly limited to, acupressure and reflexology applications wherein an individual desires to administer specific pressure to his or her hand, ankle, arm, foot, ear or other appropriate site for relief from headaches, menstrual cramps, carpel tunnel, sinus pain or other body aches or pains susceptible to acupressure or reflexology treatment.

### BACKGROUND OF THE INVENTION

Non-medicinal alternatives for health maintenance and wellness are becoming increasingly popular. As a result, the marketplace has become crowded with devices intended to assist the user's well being, such as magnetic bracelets, crystals and therapeutic herbal wraps and pillows. Alternative remedies once utilized by only small segments of the population have now become mainstream. Acupressure and reflexology are examples of such remedies.

Acupressure techniques originated in ancient China and are founded on the theory that the energy balance in the body can be enhanced by the stimulation of particular points. Reflexology techniques were utilized in ancient Egypt, India, China and Japan and depend upon the idea that the organs and systems for all body functions can be influenced through stimulation of points on the soles of the feet and the palms of the hands. Both acupressure and reflexology utilize the application of pressure to stimulate particular points on the body.

It is believed that energy, or chi, flows through meridian pathways that interconnect body points with the brain and specifically associated organs. Stimulating meridian points with acupressure is intended to release chi, enabling energy to flow unobstructed, thus promoting the production and release of endorphins, the body's naturally generated painkillers, to provide pain relief for the associated organ.

Pressure is usually applied at a specific meridian point by the thumbs, knuckles or fingers for approximately two minutes. For example, pressure applied to the fleshy area between the thumb and index finger can relieve a tension headache and/or constipation. Pressure at a point located two finger widths up from the crease of the wrist can act as an appetite suppressant. The center of the crease behind the knee can provide relief from lower back pain, if pressure is applied thereto.

Reflexology is believed to stimulate natural healing energy in the body, initiating electrochemical responses. Pressure applied to points on the feet and hands correlate to particular body areas. The inner edges of the feet reflex the spine, with the distance along the edge of the foot correlating proportionally to the distance of the targeted area along the spine. Toes and fingers reflex the head, and the area just below the toes reflexes the neck and shoulders. Unlike acupressure, wherein knowledge of meridian location and linkage is necessary, an untrained individual can easily find and manipulate the reflex point that will alleviate a specific pain since areas needing work are typically sensitive.

Devices have been introduced to assist professionals in the application of both acupressure and reflexology techniques, as well as to aid self-administering individuals. Many known acupressure devices are designed for utilization at one specific location. One commonly targeted area is the ear. Ring type devices having a spring clamp-type action have been described, wherein the pressure delivery thereof is a function of how hard the user squeezes a pliant plastic or metal apparatus. Others employ a simple threaded screw-type mechanism, much like that of a vice, to supply pressure adjustment.

In addition to those for the ear, devices have been described for utilization on the hand, wrist, foot and toe. A common design of these devices is the threaded screw-type mechanism, wherein one pressure delivery point is threaded to enable the user to rotatably adjust the pressure delivered thereby. Another design incorporates a spring-loaded hinge, much like that commonly utilized for hair and utility clips. The pressure delivered by this type of device is dependent upon the spring coefficient and is not readily adjustable by the user. Sometimes the user can apply additional clamping force to increase the pressure. In fact, some devices describe locking clips that can be positioned to hold such increased compression.

However, successful treatment with acupressure and reflexology is not only dependent upon the proper location for the application of pressure, but requires the utilization of the correct force. Too little pressure can fail to stimulate the meridian or reflex point. Too much pressure can increase pain and even cause injury. Uncontrollable variances and unpredictable forces necessarily and disadvantageously result from utilization of the aforementioned pressure adjustment mechanisms.

Recognizing the need for more specific pressure adjustment, some devices have been designed to incorporate the threaded screw-type mechanism with a spring-loaded or ratcheted hinge. However, while these combination-type devices do provide increased pressure adjustability, the force of the applied pressure is neither specifically selectable nor reproducible.

Furthermore, even if reproducibly specific pressures could be selected, since body points vary in size and shape, a variety of pressure application tips or pads is desirable. While some devices recognize the importance of pressure applicator shape and contemplate differences thereof, each is disadvantageous in view of the present invention in that a separate device would need to be purchased for each type of pressure application tip desired.

Therefore, it is readily apparent that there is a need for an acupressure and reflexology clamping apparatus capable of delivering incrementally variable and reproducible quantities of pressure to specific body locations and maximizing the effective relief experienced at each such location by utilizing a variety of easily interchangeable pressure delivery tips, thus preventing the above-discussed disadvantages.

### BRIEF SUMMARY OF THE INVENTION

Briefly described, in a preferred embodiment, the present invention overcomes the above-mentioned disadvantages, and meets the recognized need for such a device, by providing an acupressure/reflexology clamp device enabling an individual to self-administer direct, adjustable and reproducible pressure to selected body points to provide pain relief.

According to its major aspects and broadly stated, the present invention is an acupressure and reflexology clamping apparatus capable of delivering incrementally variable and reproducible quantities of pressure to specific body locations and maximizing the effective relief experienced at each such location by utilizing a variety of easily interchangeable pressure delivery tips.

More specifically, the present invention in its preferred form is a plastic or metal “C-shaped” type clamp with a centrally positioned adjustable hub, or hinge, and interchangeable pressure delivery tips. The preferred hub has a ratcheting mechanism to enable fine incremental adjustment of pressure settings and locking thereof. User controlled movement of the notched hinge provides precise decreases, preferably in millimeter increments, of spacing between the pressure delivery tips, thereby increasing the degree of pressure applied to the selected body point. Quick release features are also provided to enable the user to quickly cease application of the pressure.

Several interchangeable tips are provided to enable user selection thereof for a variety of applications and body points. The variety of pressure delivery tips includes graduated-size tips having smooth, rounded, substantially circular shapes, substantially conical shapes, substantially spherical shapes and/or other shapes suitable for utilization in the application of acupressure or reflexology treatment. Magnetic tips are also provided to enable magnetic healing therapy application. Each tip is removably positioned at the peripheral end of the “C-shaped” clamp, either with a button-lock, magnetic lock, elasticized sleeve mechanism or threads.

In addition, a variety of clamp sizes are also provided to accommodate acupressure application on a user's hand, ankle, arm, ear or other appropriate selected pain relief site for headaches, premenstrual and menstrual cramps, carpel tunnel, sinus pain and/or any other body aches or pains susceptible to acupressure/reflexology relief, wherein pressure delivery tips are easily interchangeable therebetween.

In use, appropriately sized and shaped pressure delivery tips are user-selected and mounted to the clamp. The acupressure/reflexology device is secured to a body point, such as the fleshy area of the hand between the thumb and index finger. The clamp is adjusted in millimeter increments until the desired amount of pressure is being applied to the body point. Treatment continues for approximately one to two minutes, wherein pain relief should begin to be realized. The user then releases the clamp lock and removes the device from the body point. If additional treatment areas are desired, tips are interchanged as appropriate and the clamping device is reapplied to the new body point location.

A feature and advantage of the present invention is the ability of such a device to enable specific and incremental adjustment, wherein pressure delivered thereby is accurate and reproducible.

A feature and advantage of the present invention is the ability of such a device to provide easily interchangeable pressure delivery tips in a variety of shapes and sizes.

A feature and advantage of the present invention is, the ability of such a device to provide an accurate method for self-application of acupressure and/or reflexology treatment.

A feature and advantage of the present invention is the ability of such a device to deliver reflexology and acupressure treatment to specific body locations.

A feature and advantage of the present invention is the ability of such a device to assist in the delivery of magnetic healing treatments.

A feature and advantage of the present invention is the ability of such a device to provide quick, non-medicinal pain relief from headaches, menstrual cramps, carpel tunnel, sinus pain and/or other body aches or pains susceptible to acupressure or reflexology treatment.

A feature and advantage of the present invention is that the clamp and locking hub may incorporate a variety of colors, styles and indicia.

These and other objects, features and advantages of the invention will become more apparent to one skilled in the art from the following description and claims when read in light of the accompanying drawings.

### BRIEF DESCRIPTION OF THE DRAWINGS

The present invention will be better understood by reading the Detailed Description of the Preferred and Alternate Embodiments with reference to the accompanying drawing figures, in which like reference numerals denote similar structure and refer to like elements throughout, and in which:

FIG. 1 is a perspective view of an acupressure and reflexology clamp apparatus according to a preferred embodiment of the present invention, showing a first interchangeable pressure delivery tip releaseably locked thereon and a second interchangeable pressure delivery tip removed therefrom.

FIG. 2 is a perspective view of preferred interchangeable locking pressure delivery tip styles for utilization with the acupressure and reflexology clamp apparatus of FIG. 1.

FIG. 3 is a perspective view of an acupressure and reflexology clamp apparatus according to an alternate embodiment of the present invention, showing a first interchangeable pressure delivery tip threadedly secured therein and a second interchangeable pressure delivery tip removed therefrom.

FIG. 4 is a perspective view of interchangeable threaded pressure delivery tip styles for utilization with the acupressure and reflexology clamp apparatus of FIG. 3.

FIG. 5 is a perspective view of an acupressure and reflexology clamp apparatus according to an alternate embodiment of the present invention, showing a first pressure delivery tip with an interchangeable tip elastically secured thereon and a second pressure delivery tip with an elasticized interchangeable tip removed therefrom.

FIG. 6 is a perspective view of interchangeable elasticized pressure delivery tip styles for utilization with the acupressure and reflexology clamp apparatus of FIG. 5.

### DETAILED DESCRIPTION OF THE PREFERRED AND ALTERNATE EMBODIMENTS

In describing the preferred and alternate embodiments of the present invention, as illustrated in the figures, specific terminology is employed for the sake of clarity. The invention, however, is not intended to be limited to the specific terminology so selected, and it is to be understood that each specific element includes all technical equivalents that operate in a similar manner to accomplish similar functions.

Referring now to FIG. 1, the present invention is an acupressure/reflexology clamping device 10 comprising first arm 20, second arm 40, hub 60 and a pair of pressure delivery tips 80. Preferably, first arm 20 and second arm 40 are defined by first arcuate member 22 and second arcuate member 42, respectively.

First arm 20 and second arm 40 have proximate ends 24 and 44 and distal ends 26 and 46, respectively, wherein each proximate end 24 and 44 is preferably pivotally held by hub 60. Preferably, the positioning of first arm 20 and second arm 40 with respect to hub 60 defines substantially “C”-shaped acupressure/reflexology clamping device 10. Upon substantially complete compression of acupressure/reflexology clamping device 10, distal ends 26 and 46 of arms 20 and 40, respectively, are positioned adjacent to or in direct contact with one another, thereby defining a substantially circular shape. One skilled in the art would readily recognize that while a circular shape is preferred, other shapes could also be utilized, such as, for exemplary purposes only, a triangular shape or a diamond shape, wherein first arm 20 and second arm 40 could have angles defined therein.

First arm 20 and second arm 40 have inner surfaces 28 and 48 and outer surfaces 29 and 49, respectively, wherein inner surfaces 28 and 48 preferably define central apertures 30 and 50, respectively, proximate to distal ends 26 and 46, respectively. Preferably, central apertures 30 and 50 are dimensioned to receive a pair of pressure delivery tips 80, wherein the depth of central apertures 30 and 50 within first arm 20 and second arm 40, respectively, is at least sufficient to enable positioning of pressure delivery tips 80 therein. One skilled in the art would readily recognize that central apertures 30 and 50 could be defined with a depth greater than that necessary to enable positioning of pressure delivery tips 80 therein, such as, for exemplary purposes only, a depth extending within the substantially full length of first arm 20 and second arm 40, respectively, or some lesser portion than the full length thereof.

Preferably, first arcuate member 22 and second arcuate member 42 are plastic. However, other appropriate lightweight, strong materials could also be utilized, such as, for exemplary purposes only, fiberglass, aluminum, or other metal. Preferably, proximate to distal ends 26 and 46, outer surfaces 29 and 49 of first arm 20 and second arm 40, respectively, define substantially square-shaped cross-sections 32 and 52, respectively, and proximate to proximal ends 24 and 44, outer surfaces 29 and 49 of first arm 20 and second arm 40, respectively, define substantially rectangular-shaped cross-sections 33 and 53. Thus, preferably, the overall cross-sectional shape and outer dimensions of first arm 20 and second arm 4C range from substantially square-shaped cross-sections 32 and 52 at distal ends 26 and 46, respectively, gradually and incrementally changing to substantially rectangular-shaped cross-sections 33 and 53 at proximal ends 24 and 44, respectively. One skilled in the art would readily recognize, however, that any cross-sectional shape could be utilized without departing from the intended scope of the present invention.

Proximal ends 24 and 44 of first arm 20 and second arm 40, respectively, are preferably pivotally positioned within hub 60. Preferably, hub 60 has first substantially circular-shaped, substantially flat surface 62, second substantially circular-shaped, substantially flat surface 64 and peripheral wall 66. Preferably, peripheral wall 66 has substantially rectangular-shaped apertures 68a and 68b defined therein. Preferably, apertures 68a and 68b are dimensioned to receive and allow lengthwise pivotal movement of substantially rectangular-shaped proximal ends 24 and 44, respectively, of first arm 20 and second arm 40, respectively, therein. One skilled in the art would readily recognize that, while a substantially rectangular shape is preferred for apertures 68a and 68b, peripheral wall 66 of hub 60 could have a substantially continuous, substantially centrally positioned channel to receive and allow lengthwise pivotal movement of substantially rectangular-shaped proximal ends 24 and 44, respectively, therein.

Hub 60 preferably has a ratchet mechanism known in the art, wherein incrementally adjustable positioning and locking thereof is enabled. Preferably, each incremental position adjustment of the known ratchet mechanism corresponds to a millimeter change in the relative positions of first arm 20 and second arm 40, wherein each adjustment results in a one millimeter decrease in the distance between distal end 26 of first arm 20 and distal end 46 of second arm 40. One skilled in the art would readily recognize that, while one millimeter is the preferred incremental adjustment factor, other factors could be utilized, such as, for exemplary purposes only, those ranging from one micrometer to one centimeter, wherein smaller factors would increase and larger factors would decrease fine pressure adjustment capabilities.

Preferably, visual gauge 70 is provided on first surface 62 of hub 60, wherein ratchet adjustment selections are indicated thereby. In the preferred form, visual gauge 70 comprises viewing window 72, indicator needle 74 and ratchet position indicator markings (not shown). Preferably, ratchet position indicator markings are numbers, however, one skilled in the art would readily recognize that other markings could be utilized, such as, for exemplary purposes only, letters, symbols or color coding. In addition, while the preferred form of the invention provides viewing window 72 and indicator needle 74, thereby enabling reproducible ratchet adjustment selection viewing, other means of selection viewing could be utilized, such as, for exemplary purposes only, a limited viewing window wherein selected measurement settings are displayed and/or a mechanical click-type counter known in the art.

Ratchet hub release activator 76 is known in the art, is preferably centrally positioned within hub 60 and is accessible from first surface 62 thereof, whereby user depression of ratchet hub release activator 76 unlocks hub 60, enabling first arm 20 and second arm 40 to pivotally move, increasing the distance between distal end 26 of first arm 20 and distal end 46 of second arm 40, thereby returning acupressure/reflexology clamping device 10 to the uncompressed position.

Central aperture 30 of distal end 26 of first arm 20 and central aperture 50 of distal end 46 of second arm 40 releaseably carry pressure delivery tips 82a and 82b, respectively, therein. Preferably, pressure delivery tips 82a and 82b are defined by substantially rectangular-shaped locking bases 84a and 84b, respectively, and substantially rounded pressure delivery pads 86a and 86b.

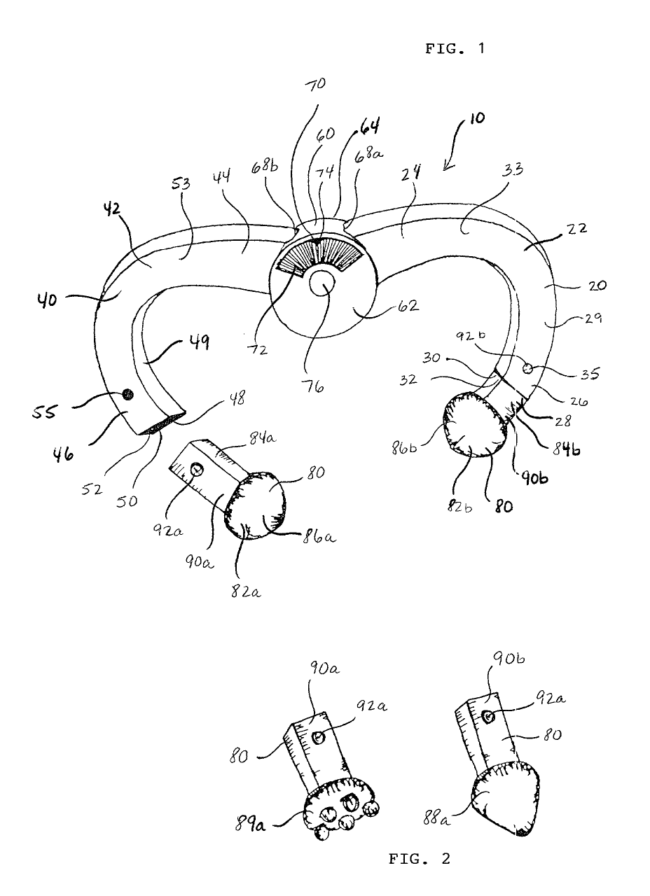

As best seen in FIGS. 1 and 2, several interchangeable locking pressure delivery tip styles are preferably provided, wherein a user selects between substantially rounded pressure delivery pads 86a and 86b, substantially conical pressure delivery pads 88a and 88b (88b not shown) or substantially textured pressure delivery pads 89a and 89b (89b not shown). One skilled in the art would readily recognize that, while the depicted and described delivery tip styles are preferred, other appropriately shaped and sized pressure delivery pads could be utilized, wherein variation of the nature or scope of pressure delivery is desired.

Substantially rectangular shaped locking bases 84a and 84b of pressure delivery tips 82a and 82b, respectively, define outer surfaces 90a and 90b thereof. Preferably, at least one pressure sensitive protuberance 92a and 92b is positioned on outer surfaces 90a and 90b, respectively. Correspondingly dimensioned apertures 35 and 55 are preferably defined within outer surfaces 29 and 49 of first arm 20 and second arm 40, respectively, proximate to distal ends 30 and 50 thereof, to receive protuberances 92a and 92b therein. Whereupon a pair of pressure delivery tips 80 is positioned within acupressure/reflexology clamping device 10, protuberance, 92a and 92b extend through apertures 35 and 55, effectively and releaseably locking pressure delivery tips 80 in place.

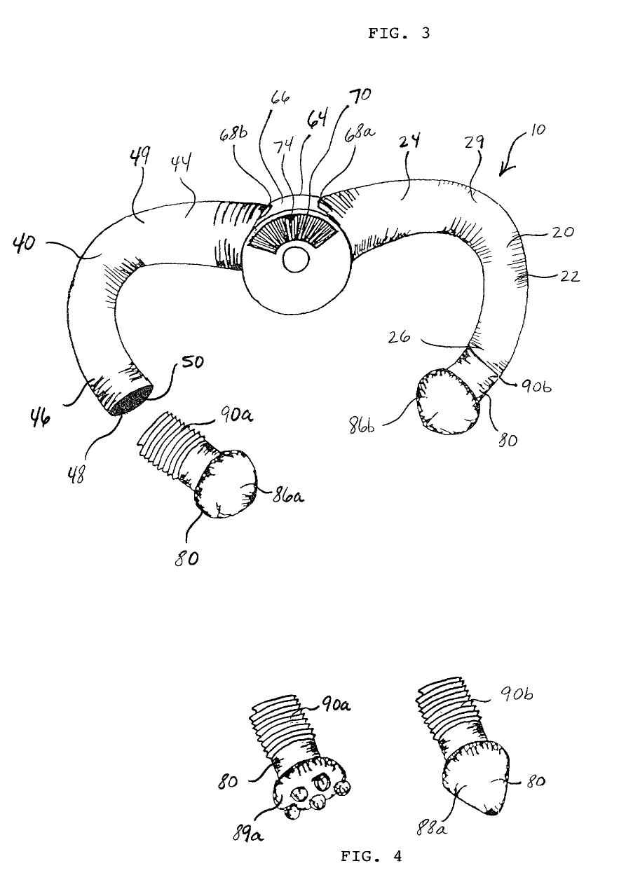

In one alternate embodiment, as best seen in FIG. 3, outer surface 29 of first arm 20 and outer surface 49 of second arm 40 could define a substantially circular or substantially oval shaped cross-section, wherein bases 90a and 90b of pressure delivery tips 80 could be correspondingly shaped.

In one alternate embodiment, as best seen in FIG. 3 and FIG. 4, outer surface 29 of first arm 20 and outer surface 49 of second arm 40 could define a substantially circular shaped cross-section, wherein inner surfaces 28 and 48, thereof, could be threaded and bases 90a and 90b of pressure delivery tips 80 could be correspondingly threaded, thereby providing a screw-type mechanism for releasable interchanging of pressure delivery tips 80, in lieu of or in addition to pressure sensitive protuberance 92.

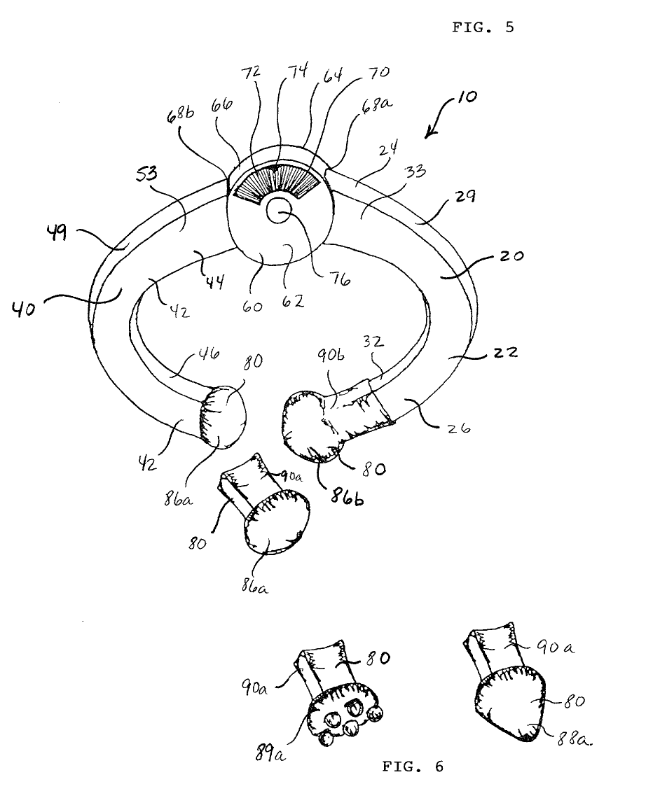

In one alternate embodiment, as best seen in FIG. 5 and FIG. 6, distal ends 26 and 46 of first arm 20 and second arm 40, respectively, could integrally define and permanently carry pressure delivery tips 80. Interchangeable pressure delivery tip styles could be provided, wherein bases 90 could be elasticized sleeves enabling secure, removable positioning over integral pressure delivery tips 80.

In one alternate embodiment, inner surfaces 28 and 48 of first arm 20 and second arm 40, respectively, could be magnetically charged or could carry a magnet therein, wherein bases 90a and 90b of pressure delivery tips 80 could carry corresponding magnets or magnetic charges to enable removably secure placement of pressure delivers tips 80 in first arm 20 and second arm 40.

In one alternate embodiment, delivery pads of pressure delivery tips 80 could carry magnets positioned wherein placement of acupressure/reflexology clamping device 10 with pressure delivery tips 80 against a selected body point could enable coincident magnetic therapy application.

In one alternate embodiment, acupressure/reflexology clamping device 10 could have an electronic or mechanical timer provided thereon, whereby a user could select a length of time for treatment and could be audibly and/or visibly notified when that period has expired.

In one alternate embodiment, first arm 20 and second arm 40 could each have a generally elongated adjustment arm or handle extending therefrom, whereby a user could easily compress acupressure/reflexology clamping device 10, wherein the generally elongated adjustment arms or handles could be separate from first arm 20 and second arm 40, could be attached thereto or could be formed integrally therewith.

In use, appropriately sized and shaped pressure delivery tips are user-selected and mounted to an appropriately sized acupressure/reflexology clamping device 10. Acupressure/reflexology clamping device 10 is secured to a body point, such as the fleshy area of the hand between the thumb and index finger. Acupressure/reflexology clamping device 10 is adjusted in preferably millimeter increments until a desired amount of pressure is being applied to the selected body point. Treatment continues for approximately one to two minutes, wherein pain relief should begin to be realized. The user then releases clamp lock 76 of hub 60 and removes acupressure/reflexology clamping device 10 from the treated body point. If additional treatment areas are desired, tips 80 may be interchanged as appropriate, and acupressure/reflexology clamping device 10 is reapplied to the new body point location.

Having thus described exemplary embodiments of the present invention, it should be noted by those skilled in the art that the within disclosures are exemplary only, and that various other alternatives, adaptations, and modifications may be made within the scope of the present invention. Accordingly, the present invention is not limited to the specific embodiments illustrated herein, but is limited only by the following claims.

What is claimed is:

1. A clamping device for acupressure or reflexology, comprising:
a first arm having a proximate end and a distal end,
a second arm having a proximate end and a distal end,
a hub, wherein said proximate end of said first arm and said proximate end of said second arm are pivotally carried thereby,
at least one pressure delivery tip,
means for carrying said pressure delivery tip on said distal end of said first arm, and
means for carrying said pressure delivery tip on said distal end of said second arm.

2. The clamping device of claim 1, further comprising means for incremental adjustment of said hub.

3. The clamping device of claim 2, wherein said means for incremental adjustment of said hub is a ratchet mechanism.

4. The clamping device of claim 2, further comprising means for releasable locking of said hub.

5. The clamping device of claim 4, wherein said means for releasable locking of said hub is a pressure-sensitive button and wherein the application of pressure to said button enables free movement of said first arm and said second arm relative to said hub.

6. The clamping device of claim 1, wherein said at least one pressure delivery tip has a pressure delivery proximate end and a mount end, wherein said first arm has a first aperture defined in said distal end thereof, wherein said first aperture is dimensioned to receive said mount end of said at least one pressure delivery tip, wherein said first aperture is said means for carrying said pressure delivery tip on said distal end of said first arm, wherein said second arm has a second aperture defined in said distal end thereof, wherein said second aperture is dimensioned to receive said mount end of said at least one pressure delivery tip, and wherein said second aperture is said means for carrying said pressure delivery tip on said distal end of said second arm.

7. The clamping device of claim 1, wherein said first arm and said second arm each have a substantially square-shape cross-section proximate to said distal end thereof, wherein said first arm and said second arm each have a substantially rectangular-shape cross-section proximate to said proximal end thereof.

8. The clamping device of claim 1, wherein said hub has a first substantially flat surface, a second substantially flat surface and a peripheral wall, wherein said peripheral wall has generally rectangular-shaped apertures, and wherein said first arm and said second arm are pivotally mounted therethrough.

9. The clamping device of claim 1, wherein said hub has a first substantially flat surface, a second substantially flat surface and a peripheral wall, wherein said peripheral wall has a substantially continuous channel defined therein, and wherein said first arm and said second arm are pivotally mounted therethrough.

10. The clamping device of claim 3, further comprising a visual indicator for ratchet adjustment selection.

11. The clamping device of claim 6, wherein said at least one pressure delivery tip is a plurality of pressure delivery tips interchangeable relative to said clamping device.

12. The clamping device of claim 11, wherein said plurality of pressure delivery tips includes a pressure delivery tip having a generally round pressure delivery proximate end.

13. The clamping device of claim 11, wherein said plurality of pressure delivery tips includes a pressure delivery tip having a generally conical pressure delivery proximate end.

14. The clamping device of claim 11, wherein said plurality of pressure delivery tips includes a pressure delivery tip having a generally textured pressure delivery proximate end.

15. The clamping device of claim 6, wherein said mount end of said at least one pressure delivery tip carries at least one pressure sensitive protuberance, and wherein said distal end of said first arm and said distal end of said second arm each carry at least one protuberance receptacle.

16. The clamping device of claim 6, wherein said mount end of said at least one pressure delivery tip is generally circular shaped and threaded, wherein said first arm and said second arm each have a substantially circular cross-section, and wherein said distal end of said first arm and said distal end of said second arm are correspondingly threaded.

17. The clamping device of claim 1, wherein a first said at least one pressure delivery tip is integrally formed on said distal end of at least one said arm and a second said at least one pressure delivery tip has a generally resilient mount end as means for carrying said pressure delivery tip.

18. The clamping device of claim 6, wherein said mount end of said at least one pressure delivery tip is magnetic and wherein said distal end of said first arm and said distal end of said second arm are correspondingly magnetic.

19. The clamping device of claim 6, wherein pressure delivery end of said at least one pressure delivery tip carries at least one magnet.

20. The clamping device of claim 1, further comprising a timing device.

21. The clamping device of claim 1, further comprising a first generally elongated handle and a second generally elongated handle, wherein compression of said first generally elongated arm and said second generally elongated arm compresses said first arm and said second arm of said clamping device.

22. A pressure delivery device, comprising:
a first arm having a proximate end and a distal end;
a second arm having a proximate end and a distal end;
a hub, wherein said proximate end of said first arm and said proximate end of said second arm are pivotally carried thereby, wherein said hub has a ratchet mechanism enabling incremental adjustment of said first arm and said second arm, and wherein said hub has a releasable locking mechanism for positioning said first arm and said second arm;
a first pressure delivery tip, said tip having a mount end removably carried by said first arm;
a second pressure delivery tip, said tip having a mount end removably carried by said second arm.

23. A method of acupressure/reflexology treatment comprising the steps of:
a) obtaining a clamping device having two pivotally carried arms, an incrementally adjustable hub, wherein said arms are pivotally carried by said hub, and a plurality of interchangeable pressure delivery tips removably carried by said arms;
b) selecting a tip from said plurality of interchangeable pressure delivery tips;
c) installing said selected tip onto said clamping device;
d) placing said clamping device proximate to a selected body point;
e) adjusting said hub until a desired amount of pressure is being applied to the selected body point;
f) releasing said hub at the conclusion of treatment.
